(19) United States
(12) Patent Application Publication
(10) Pub. No.: US 2025/0093437 A1
(43) Pub. Date: Mar. 20, 2025

(54) DEVICE FOR REDUCING MRI RF-INDUCED HEATING IN ACTIVE IMPLANTED MEDICAL DEVICE LEADS

(71) Applicant: Wavegate Corporation, Lake Charles, LA (US)

(72) Inventor: Erich W. Wolf, II, Lake Charles, LA (US)

(73) Assignee: Wavegate Corporation, Lake Charles, LA (US)

(21) Appl. No.: 18/962,745

(22) Filed: Nov. 27, 2024

Related U.S. Application Data
(63) Continuation-in-part of application No. 17/443,174, filed on Jul. 21, 2021.
(60) Provisional application No. 62/705,893, filed on Jul. 21, 2020.
(60) Provisional application No. 63/602,872, filed on Nov. 27, 2023.

(51) Int. Cl. G01R 33/28; H01F 27/08; H01M 4/04; H01M 4/66

(52) U.S. Cl. CPC G01R 33/288; H01F 27/08; H01M 4/04; H01M 4/662

(57) ABSTRACT

The present invention relates to a device for reducing MRI-induced RF heating in active implantable medical device (AIMD) leads, particularly spinal cord stimulator leads. The device incorporates an air-core transformer to increase inductance within the lead system, significantly reducing RF coupling and localized heating at the electrode-tissue interface during MRI procedures. The transformer features a primary coil formed by the lead and a secondary coil embedded in a biocompatible toroidal bobbin, which doubles as an anchoring mechanism. The bobbin securely holds the lead while maintaining flexibility for implantation. Anchoring is achieved through shape-memory retaining pins that adapt upon deployment, ensuring positional stability. This innovative design enhances MRI compatibility without compromising AIMD functionality, providing a safer and more effective solution for managing chronic pain and other neurological conditions. By addressing both lead stability and RF heating, the device supports safer imaging for AIMD patients.

### CROSS-REFERENCE TO RELATED APPLICATIONS

This application is a continuation-in-part of U.S. application Ser. No. 17/443,174 filed on Jul. 21, 2021 which claims priority benefit from U.S. Provisional Application No. 62/705,893 filed on Jul. 21, 2020. This application claims priority benefit from U.S. Provisional Application No. 63/602,872 filed on Nov. 27, 2023. The patent applications identified above are incorporated here by reference in their entirety to provide continuity of disclosure.

### FIELD OF THE INVENTION

The present invention relates to insertion and anchoring for spinal cord stimulator percutaneous leads and to reduction of MRI heating of implanted components.

### BACKGROUND OF THE INVENTION

The use of Active Implantable Medical Devices (AIMDs), such as spinal cord stimulators, has become increasingly common in managing chronic pain and neurological conditions that are unresponsive to traditional therapies. These devices typically consist of a pulse generator, often implanted subcutaneously, connected to electrodes that interface with neural structures through leads routed to specific anatomical targets. While effective, AIMDs are subject to safety challenges, particularly when patients undergo Magnetic Resonance Imaging (MRI) due to the interaction between the device's conductive leads and MRI's strong Radio Frequency (RF) fields.

The spinal cord and surrounding anatomy provide a unique challenge for AIMD lead placement, with the dura mater covering the spinal cord, surrounded by cerebrospinal fluid, vertebrae, and musculature layers, including the thoracolumbar fascia. Leads are often secured to these muscular and fascial structures to prevent migration. However, during MRI exposure, these implanted leads may act as unintended antennas, capturing and conducting RF energy from the MRI environment. This phenomenon can cause high-voltage standing waves along the lead, creating regions of intense ohmic heating, especially at the electrode-tissue interface. Such heating can elevate tissue temperatures, with potentially dangerous outcomes if temperatures exceed 41° C., causing thermal injury to adjacent nerves, muscle, or other tissues.

The physics of RF-induced heating in AIMDs is complex, as the body's physiological structure creates a heterogeneous environment. The interaction of RF waves with implant leads can vary due to the anatomical positioning, surrounding tissue types, and MRI parameters. Key to this effect is the lead's inherent electrical properties-its length, configuration, and insulation, which influence how it couples with external RF fields. Additionally, the lead's flexibility allows it to curve through the body, increasing the likelihood of capturing RF energy in non-linear pathways that amplify heating. Given these challenges, MRI use with AIMDs is often restricted or “conditionally compatible,” which mandates specific scanning parameters to limit heating but may lead to suboptimal imaging quality.

The prior art has explored various solutions to mitigate RF-induced heating. For example, U.S. Pat. No. 9,302,101, to Wahlstrand, et al. discloses MRI-induced heating by incorporating a lead structure that conducts RF energy from the lead to surrounding tissue, dispersing it along the lead length to reduce localized heating, particularly at the electrode-tissue interface. While effective, this approach does not focus on altering the lead's inductance to reduce RF coupling across the entire system. In contrast, the present invention introduces an air-core transformer to significantly increase inductance within the lead. This configuration directly reduces RF coupling and mitigates heating without relying on RF conduction through surrounding tissues, thereby enhancing MRI compatibility and reducing heating risks for a wider range of implanted leads.

As another example, U.S. Pat. No. 9,061,132, to Zweber, et al. discloses reduced RF-induced heating by positioning a conductor around the exterior of an implantable device to lower current flow at the electrode-tissue interface. Although this design reduces heating near the device, it requires external positioning of the conductor and is primarily limited to device housing. The present invention, however, increases inductance within the lead system itself by using an internal air-core transformer, allowing RF coupling reduction directly along the lead path without relying on external conductors. This innovation provides a more adaptable solution applicable to the lead itself, enhancing MRI safety across diverse implant locations and configurations.

U.S. Pat. No. 8,311,643, to North provides an anchoring device specifically designed for spinal cord stimulator leads, featuring a central lumen for fitting the lead and a secondary lumen for adhesive injection to prevent lead migration. Although effective for positional stability, this design does not address MRI-induced heating. The present invention combines anchoring with MRI heating reduction by integrating an air-core transformer within the anchor design, increasing lead inductance and reducing RF coupling. This approach not only stabilizes the lead position but also offers protection against MRI-related heating, providing a dual solution that improves patient safety during MRI scans.

Despite the innovation of the prior art, a reliable solution to minimize RF-induced heating across various anatomical scenarios is still required.

### SUMMARY OF THE INVENTION

The devices and methods disclosed present a novel solution to reducing RF-induced heating in AIMD leads by increasing the lead's inductance to decrease RF coupling. Specifically, it introduces an air-core transformer configuration within the lead system that significantly raises the effective inductance of the lead, resulting in a reduction in power dissipation at the electrode-tissue interface. This increase in inductance is achieved through enhanced mutual inductance between the primary and secondary coils of the air-core transformer. The coaxial alignment of the coils ensures optimal electromagnetic coupling, which amplifies the inductive effect. This configuration not only reduces the RF energy transfer into the lead but also improves the overall efficiency of the transformer by minimizing energy losses. By leveraging mutual inductance, the design addresses the inherent challenges of MRI environments, significantly reducing the potential for localized heating. This solution provides a safer alternative for MRI compatibility in AIMDs by addressing heating without compromising the lead's integrity, flexibility, or functionality.

The device is structured around a primary and secondary coil air coil transformer. The primary coil is created by winding one or more loops of the AIMD lead around a cylindrical form or bobbin. This primary coil acts as part of the air-core transformer, with the secondary coil situated coaxially within the primary. The secondary coil, constructed from biocompatible materials like platinum or copper, is shorted, which enhances the mutual inductance between the two coils. The higher inductance effectively reduces RF coupling into the lead array, mitigating heating at the electrode-tissue interface.

The air-core design offers several advantages within the MRI environment. Traditional transformers rely on ferromagnetic cores, but in MRI, any ferromagnetic material would produce image artifacts and could be hazardous due to magnetic forces. By contrast, an air-core configuration ensures that the device remains non-ferromagnetic, suitable for MRI use, while still providing substantial mutual inductance to reduce RF coupling.

An integral advantage of the device is its adaptability for specific anatomical locations, particularly in spinal cord stimulator systems where the lead is secured at the point of exit through the dorsal fascia. The bobbin serves the dual purpose of both the transformer core and a secure anchoring point for the lead, maintaining its positioning and reducing the risk of migration. This configuration is particularly beneficial in spinal cord stimulation, where precise electrode positioning relative to the spinal cord is essential for effective therapy.

The device's structure includes several novel components designed to hold and stabilize the lead within the bobbin. The toroidal bobbin allows for a single or multiple lead loops, maintaining both lead flexibility and tensile strength. The slightly oversized annular slot ensures that the lead can still slide along the bobbin as needed during device insertion but can be securely locked in place when the bobbin is anchored to the fascia. Lead fixation is essential in the dynamic environment of spinal cord stimulators, where body movement and tissue changes can alter the lead's position. The secondary coil embedded within the bobbin is fabricated from a low-resistance material with multiple windings, maximizing the inductance effect.

Upon deployment, the device can be positioned at the fascia or other body anchoring points, such as along muscle layers. Retaining pins, constructed from shape-memory materials like Nitinol, are used to secure the bobbin to the body tissue. In one embodiment, these pins, initially straight at room temperature, deform or “warp” into a curved shape upon reaching body temperature, creating a spring force that anchors the device to the fascia. In another embodiment, a monopolar cautery is used to warp the retaining pins into position by transiently inducing heating of the retaining pins through application of the cautery current. This retaining pin design reduces the risk of migration and maintains the air-core transformer alignment to maximize inductance, while also allowing for ease of implantation.

The device's potential to reduce RF-induced heating has been demonstrated through simulations. For example, a typical AIMD lead with a single loop can increase inductance to 2.4 pH when paired with a 30-turn secondary coil, as opposed to 2.3 nH without the device. In a 3 T MRI scanner operating at 128 MHz, this configuration can reduce the voltage at the electrode-tissue interface by approximately 28%, translating to a nearly 50% reduction in thermal power dissipation. This reduction significantly improves MRI compatibility, allowing patients to undergo safer imaging procedures without compromising AIMD functionality.

In summary, the device and method disclosed represent significant advancements in MRI-compatible AIMD technology. By incorporating an air-core transformer that increases inductance within the lead, the device reduces RF-induced heating while also serving as a secure anchoring mechanism. This approach mitigates safety risks associated with MRI procedures for AIMD patients, broadening the applicability of AIMDs and enhancing diagnostic imaging options for individuals with implanted devices.

### BRIEF DESCRIPTION OF THE DRAWINGS

In the detailed description of the preferred embodiments presented below, reference is made to the accompanying drawings.

FIG. 1A is an isometric view of a preferred deployment tool, attached to a bobbin, in a “deployed” configuration.

FIG. 1B is a cutaway view of a preferred deployment tool, attached to a lock cap in a “loaded” configuration.

FIG. 2 is an isometric view of a preferred plunger.

FIG. 3 is an isometric view of a preferred guide channel.

FIG. 4 is an isometric view of a preferred bobbin.

FIG. 5A is an isometric view of a preferred toroidal base.

FIG. 5B is an isometric view of a preferred secondary coil.

FIG. 5C is a cross-sectional side view of a preferred secondary coil.

FIG. 6A is an isometric top view of a preferred locking cap.

FIG. 6B is an isometric bottom view of a preferred locking cap.

FIG. 7A is a side view of a preferred bobbin.

FIG. 7B is a cutaway cross-section view of a preferred bobbin.

FIG. 8A is an isometric view of a preferred retaining block.

FIG. 8B is a cutaway cross-section view of a preferred retaining block.

FIG. 8C is an isometric view of a preferred retaining block.

FIG. 8D is a flowchart of a preferred method of retaining pin preparation.

FIG. 8E is an isometric view of a preferred retaining pin in its warped configuration.

FIG. 8F is an isometric view of a preferred retaining pin in its straight configuration.

FIG. 9A is a side view of a preferred set of forceps.

FIG. 9B is a detail view of a preferred forceps tip.

FIG. 9C is a cutaway cross-section view of a preferred forceps tip.

FIG. 9D is a detail view of a preferred forceps tip.

FIG. 10A is a flow chart of a preferred method.

FIG. 10B is an isometric exploded view of a preferred deployment tool.

FIG. 10C is a side view of a preferred set of forceps and bobbin in use.

FIG. 10D is a detail isometric view of a preferred deployment tool and bobbin in use.

FIG. 10E is a detail isometric view of a preferred deployment tool and bobbin in use.

FIG. 10F is an isometric view of a preferred bobbin in an anchored configuration.

### DETAILED DESCRIPTION OF THE INVENTION

In the description that follows, like parts are marked throughout the specification and figures with the same numerals, respectively. The figures are not necessarily drawn to scale and may be shown in exaggerated or generalized form in the interest of clarity and conciseness. Unless otherwise specified, all uses of the terms “about” and “approximately” refer to a tolerance of +20%.

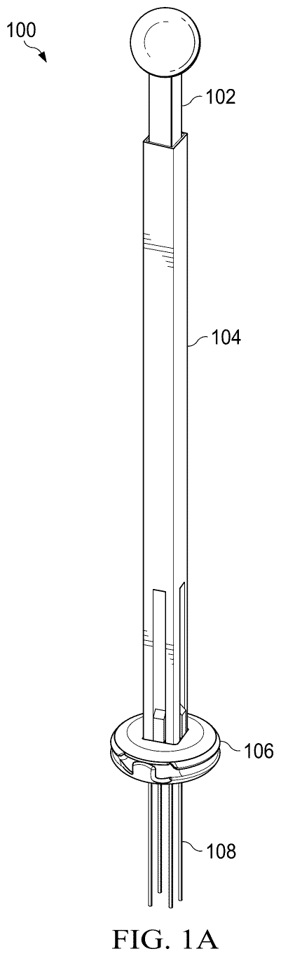

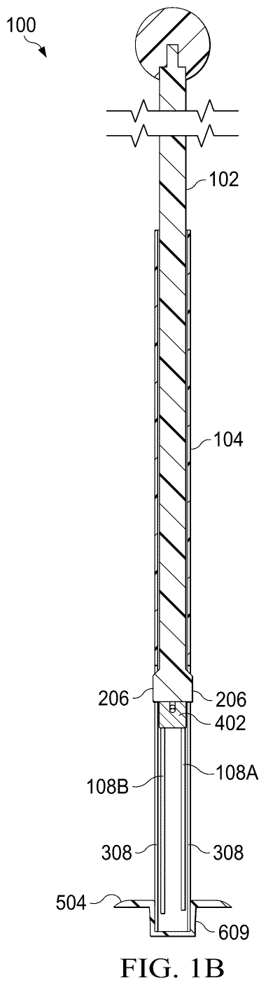

Referring then to FIG. 1A, deployment tool 100 in a “deployed” configuration will be further described. Deployment tool 100 includes plunger 102 movably disposed within guide channel 104. Guide channel 104 removably supports bobbin 106 from which retaining pins 108 protrude.

Referring also to FIG. 1B, deployment tool 100 in a “loaded” configuration will be further described.

When loaded, deployment tool 100 centrally houses retaining block 402 beneath and adjacent to plunger 102. Retaining block 402 is held within guide channel 104 by a friction fit of retaining pins 108A and 108B against the internal corners of guide channel 104. In “loaded” configuration, locking tabs 206 of plunger 102 can be seen to extend through guide slots 308 of guide channel 104.

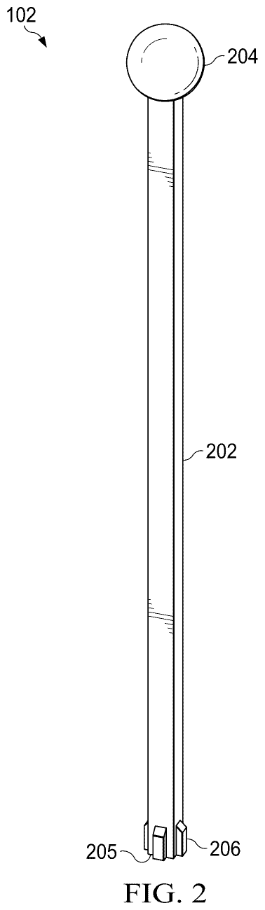

Referring then to FIG. 2, plunger 102 will be further described.

Plunger 102 is further comprised of shaft 202 terminated by button 204 and locking tabs 206. Shaft 202 preferably has a generally square or rectangular cross-section. Button 204 preferably is spherical and is attached to the shaft with a threaded connection. Shaft 202 includes at base 205 four integrally formed locking tabs 206. One locking tab is positioned on each side of shaft 202. Each locking tab is generally rectangular, designed to engage a hole in the guide channel in order to releasably hold the plunger in a specific position in the guide channel, as will be further described. Preferably, plunger 102 is a rigid thermoplastic such as polyethylene, polypropylene, polymethylmethacrylate, polyvinyl chloride, polyamide, or acrylonitrile butadiene styrene (ABS). The square shape of cross-section of shaft 202 is not mere design choice, but is important to prevent rotation of the shaft within the guide channel and rotation of the bobbin during surgery, as will be further described.

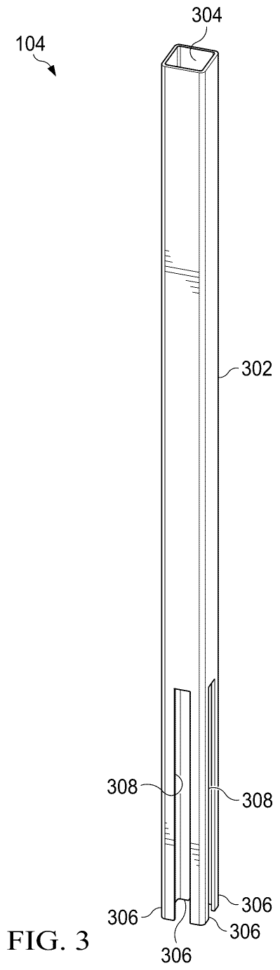

Referring then to FIG. 3, guide channel 104 will be further described.

Guide channel 104 is generally comprised of receiver tube 302 designed to slidingly accommodate shaft 202. The receiver tube preferably has a square or rectangular cross-section. Receiver tube 302 also includes center channel 304, which is also preferably square or rectangular. Receiver tube 302 at its base further comprises four connector posts 306, one each at each vertical corner. Each connector post has an “L” shaped cross section designed to frictionally engage and hold the bobbin, as will be further described. The connector posts are separated by four guide slots 308, equally dispersed around the perimeter of the receiver tube, one at the base of each flat face of the receiver tube. Preferably, receiver tube 302 is comprised of a light aluminum alloy, such as 1000, 3000, 5000, or 7,000 series aluminum alloys.

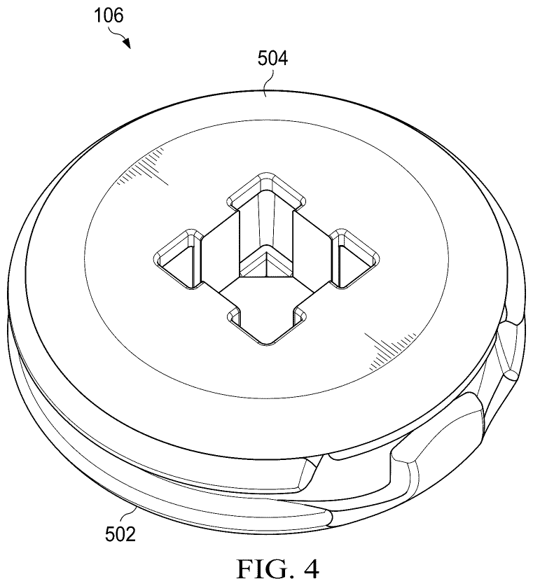

Referring then to FIG. 4, bobbin 106 will be further described. Bobbin 106 is comprised of toroidal base 502 and locking cap 504. Bobbin 106 preferably has a diameter of between about 12 millimeters and about 20 millimeters and has a depth of between about 3 millimeters and about 6 millimeters.

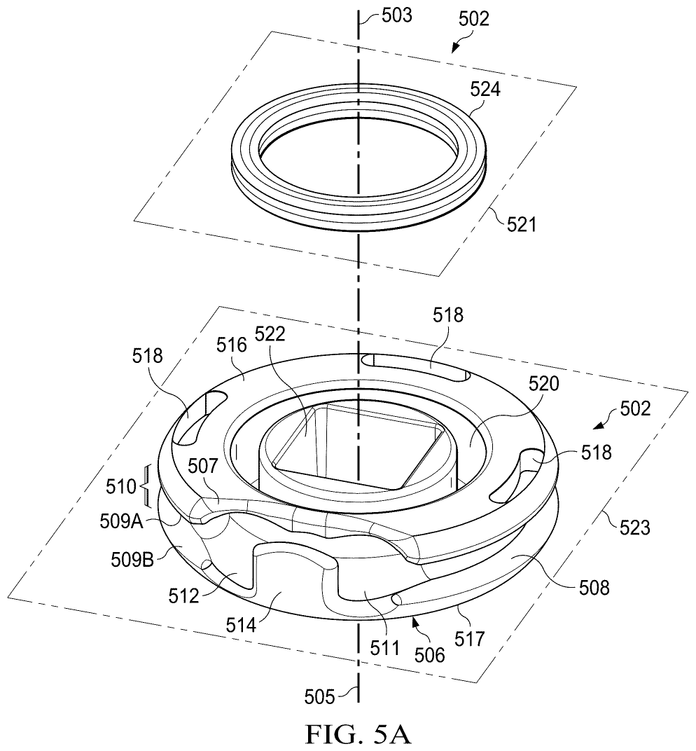

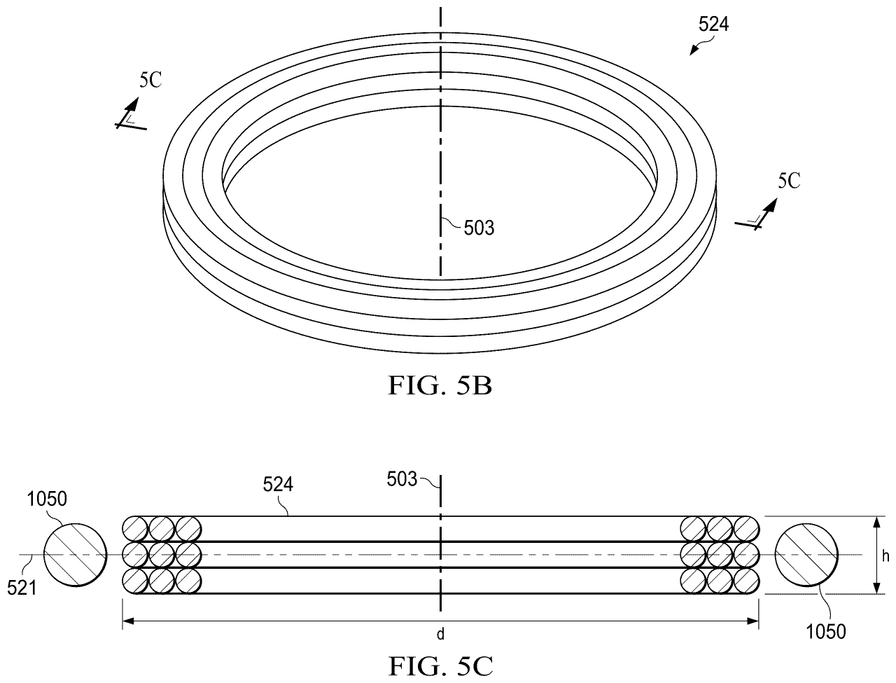

Referring then to FIG. 5A, toroidal based 502 will be further described.

Toroidal base 502 includes body 506. Body 506 has a longitudinal central axis 505 and is bisected by a transverse center plane 523. Body 506 is generally toroidal, having the features of a central axial hole 522, circumferential annular slot 508 and axial cylindrical recess 520. Preferably, hole 522 is either square or rectangular, matching the cross-sectional shape of the guide channel and plunger.

Annular slot 508 has a generally circular cross-section. In a preferred embodiment annular slot 508 has diameter slightly larger than the cross-section diameter of the implanted lead with which it will be used. Typically, the diameter of the circular cross-section ranges between about 1.2 millimeters and about 2 millimeters. The range can vary slightly depending on the manufacturer and design specifications of the lead as well as the lead type used (e.g., percutaneous leads versus paddle leads). Annular slot 508 is bounded by upper retaining lip 509A and lower retaining lip 509B. The retaining lips are also annular and form annular gap 510. Annular gap 510 is approximately 20% smaller than the diameter of annular slot 508. Typical ranges for the annular gap are between about 1 millimeter and about 1.8 millimeters. Importantly, the size of the annular gap is not mere design choice, but is designed to allow the implanted lead to be inserted into the annular slot during surgery and to allow the lead to “slip” around the circumference of the bobbin without escaping the annular slot so as to allow the bobbin to be moved along the lead, as will be further described.

Annular slot 508 is terminated by lead opening 511 and lead opening 512. Between lead opening 511 and lead opening 512 is rigid lead guide post 514. The lead openings allow the lead to enter and exit the bobbin, the lead crossing its own path behind the guide post, as will be further described.

Body 506 is bounded by top surface 516 and bottom surface 517. Both top surface 516 and bottom surface 517 are generally planar, generally parallel to each other and generally perpendicular to longitudinal central axis 505. Impressed in top surface 516 are three locking slots 518. The locking slots extend from top surface 516 and into annular slot 508. Each locking slot is semi-circular and is equally offset from the perimeter of the toroidal base. Preferably, locking slots 518 are generally parallel to longitudinal central axis 505 of body 506 and perpendicular to top surface 516 and bottom surface 517. The locking slots are preferably positioned at about 90° radial angles around the perimeter of the toroidal base. The 90° radial positioning of the locking slots is important because it allows the arcuate locking pins of the locking cap to exert a balanced compressive retaining force on the lead surface against the inside the annular slot which avoids crimping the lead at any one particular point, as will be further described.

Body 506 further comprises cylindrical recess 520. The cylindrical recess is generally a cylindrical cavity open at the top and closed at the bottom and is positioned such that its central axis, longitudinal central axis 503, is colinear with longitudinal central axis 505. Cylindrical recess 520 is further defined by transverse center plane 521, which horizontally bisects the height of the recess and is generally parallel with the top and bottom surfaces of the toroidal base.

Body 506 further includes hole 522. Hole 522 preferably has a square or rectangular cross-section designed to match the cuboidal feature of the locking cap, as will be further described. Hole 522 extends from top surface 516 to bottom surface 517 and is coaxial with longitudinal central axis 505. Each side of the hole is between about 3 millimeters and about 5 millimeters in length and height preferably form a cube shaped cavity.

In a preferred embodiment, body 506 is formed from polyetheretherketone (PEEK), polysulfone (PSU), polycarbonate (PC), polypropylene, polymethylmethacrylate (PMMA) or other high performance medical grade polymers.

Referring also to FIGS. 5B and 5C, secondary coil 524 resides within cylindrical recess 520. The coil is centrally embedded within the cylindrical recess such that longitudinal central axis 503 is coaxial with longitudinal central axis 505. The coaxial and centrally coplanar positioning of the primary and secondary coils maximizes mutual inductance by aligning the magnetic fields generated during MRI exposure. This alignment enhances the transformer's ability to counteract RF coupling into the lead, effectively mitigating energy transfer to the electrode-tissue interface. The multiple windings of the secondary coil further amplify this mutual inductance, creating a robust inductive barrier against RF energy. This design ensures that the primary coil, formed by the implanted lead, and the secondary coil work synergistically to achieve significant reductions in heating while maintaining the integrity and flexibility of the lead.

The coil is positioned in the cylindrical recess so that center plane 521 of the coil is coplanar with center plane 523 which bisects toroidal base 502. In this way, the center plane of the primary coil, formed by the electrode lead, such as lead 1050, is coplanar with the center plane of the secondary coil. Secondary coil 524 is preferably potted into cylindrical recess 520 with a medical grade epoxy or similar rigid adhesive.

Electrically, secondary coil 524 is a continuous insulated conductor, having its terminals shorted so as to form the secondary coil of the air core transformer. Secondary coil 524 may be composed of a biocompatible wire (such as MP35N, copper, platinum, platinum iridium, or other biocompatible metals) and may be a winding of multiple layers. Ideally, the end terminals of secondary coil 524 are welded rather than soldered. Secondary coil 524 is preferably between about 7 millimeters and about 12 millimeters in diameter and between about 1.5 millimeters and about 3 millimeters in height. Secondary coil 524 is preferably comprised of between about 5 and about 50 windings. Preferably, the coil is a 3×3 coaxial winding of diameter “d” of about 8 millimeters and a height “h” of about 2.5 millimeters of about 0.067 millimeter diameter wire corresponding to an American Wire Gauge (AWG) number of 42. Other coil windings and wire gauges may be used.

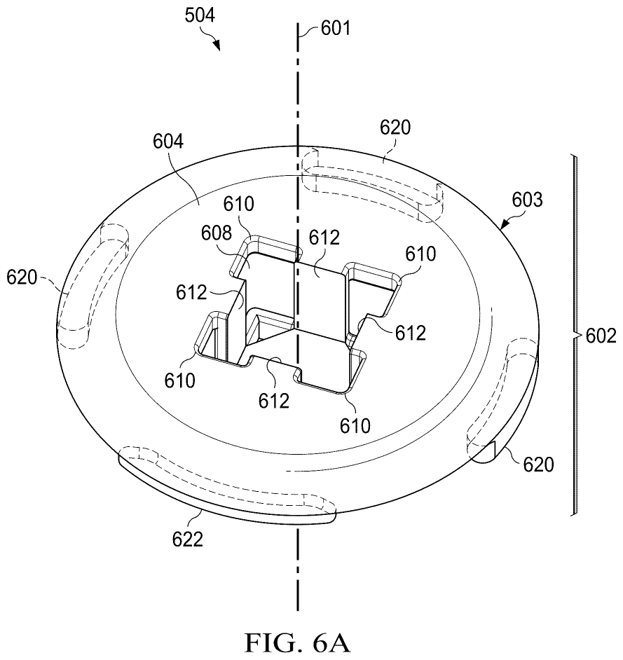

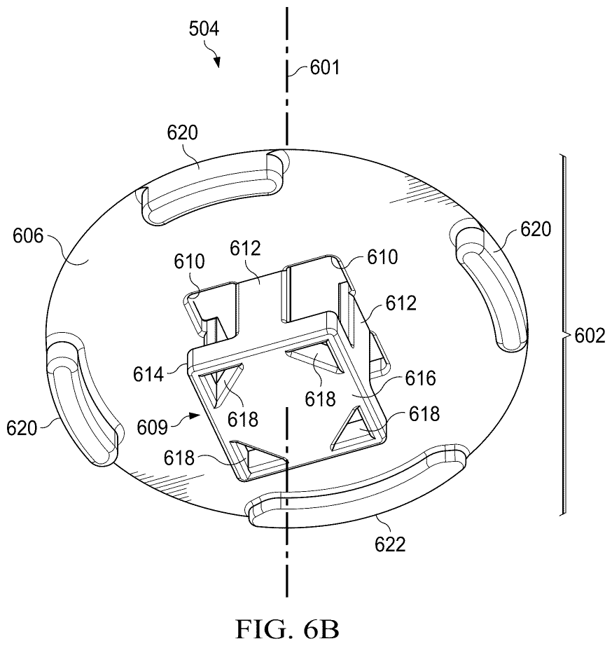

Turning to FIGS. 6A and 6B, locking cap 504 will be further described.

Locking cap 504 is comprised of body 602, having longitudinal central axis 601, and generally having upper cylinder 603 and lower cuboidal feature 609 which are integrally formed. Upper cylinder 603 includes upper surface 604 and lower surface 606. Hole 608 generally has a square or rectangular cross-section is coaxial with central axis 601, and extends from upper surface 604 to lower surface 606.

Cuboidal feature 609 is fixed within hole 608. Cuboidal feature 609 further comprises four positioning posts 612 which are attached to the inside of hole 608 and extend from upper surface 604 to perimeter wall 614 of cuboidal feature 609. Each of the positioning posts is preferably integrally formed with body 602 and perimeter wall 614. Each positioning post is centrally fixed on one flat face of hole 608. Adjacent each positioning post is a positioning indention 610. Each positioning indention extends from upper surface 604 to lower surface 606 and forms a gap sized to accommodate a single connector post of the guide channel.

Cuboidal feature 609 further comprises base 616 which directly adjoins perimeter wall 614 and is generally perpendicular each of the positioning posts. At each interior corner of perimeter wall 614, base 616 includes a triangular exit hole 618. Each triangular exit hole 618 is positioned directly below a retaining pin of the retaining block when the deployment tool is in its loaded configuration, as will be further described.

Lower surface 606 further supports three arcuate locking pins 620. The locking pins are located at approximately equal 90° radial positions around the perimeter of locking cap 504 and are designed to fit within one of locking slots 518 of toroidal base 502. When the bobbin is assembled, each arcuate locking pin proceeds downward through a locking slot and into annular slot 508, such that the locking pins exert a surface pressure on the implanted lead in the annular slot to hold the lead in place and prevent it from sliding relative to the annular slot, as will be further described.

Lower surface 606 further supports arcuate retainer tab 622. When the bobbin is assembled, the arcuate retainer tab fits within arcuate indention 507 of toroidal base 502, so as to prevent the implanted lead from escaping the accurate slot through either lead opening 511 or lead opening 512.

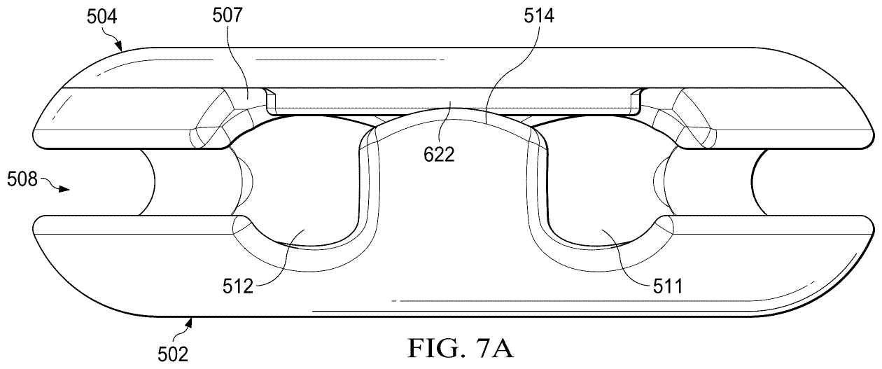

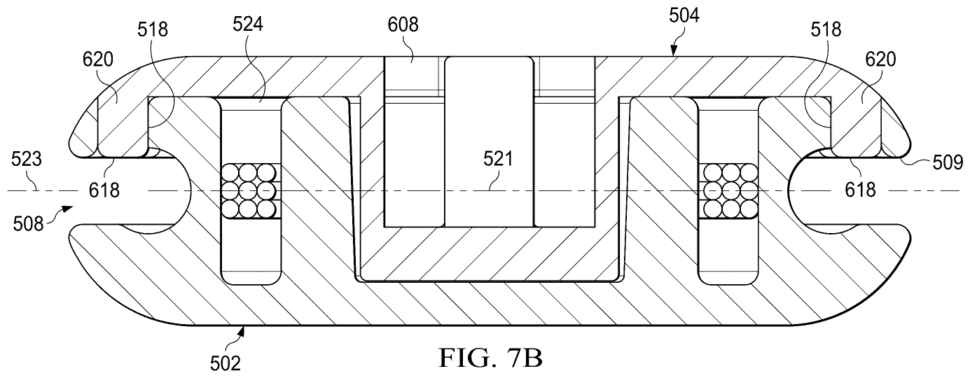

Referring then to FIGS. 7A and 7B, bobbin 106 will be further described.

When assembled, the secondary coil is potted in the cylindrical recess and the locking cap is lowered into the toroidal base. Cuboidal feature 609 is positioned in hole 522 such that base 616 is co-planar with bottom surface 517 of toroidal base 502. Arcuate retainer tab 622 extends downward adjacent lead guide post 514 and into lead opening 511 and lead opening 512, sufficient to prevent the escape of the implanted lead from annular slot 508. The accurate locking pins can be seen to extend vertically downward through the locking slots, and into annular slot 508, flush with retaining lip 509. As such, each looking tab extends into annular slot 508 approximately the depth of the retaining lip, in order to jointly clamp lead in place in the bobbin, as will be further described.

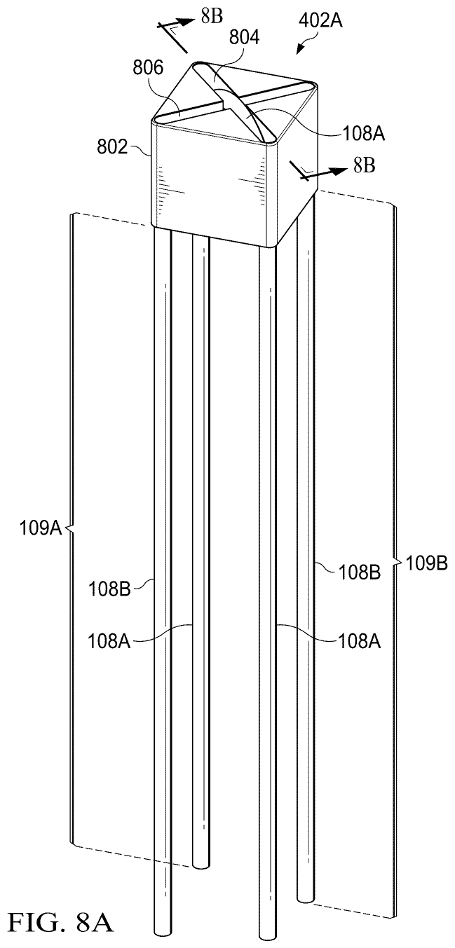

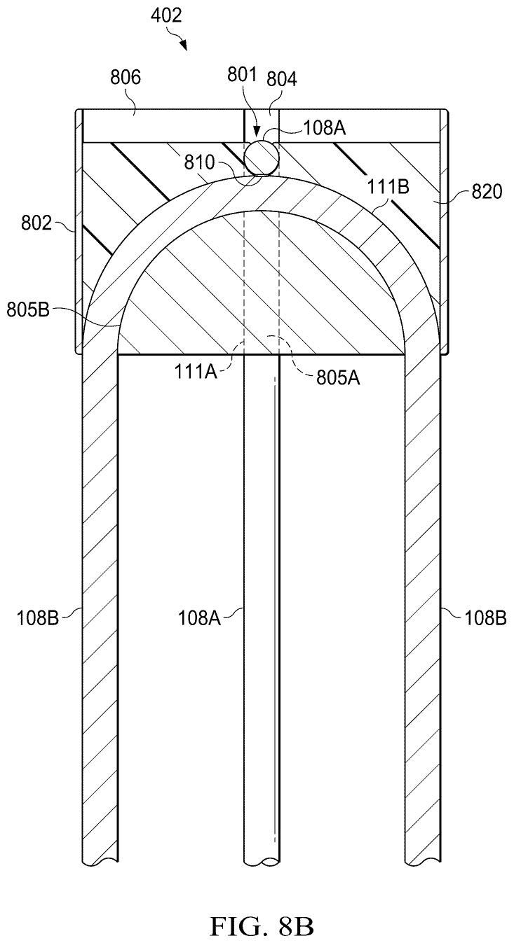

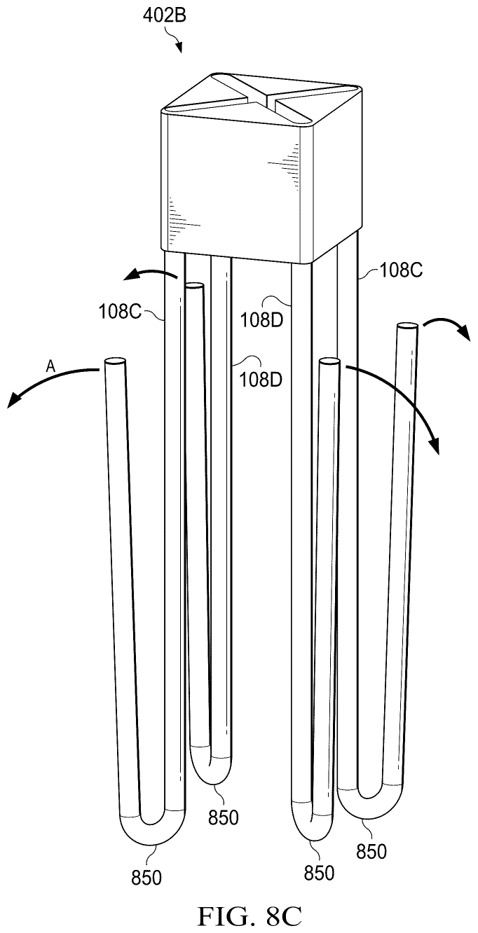

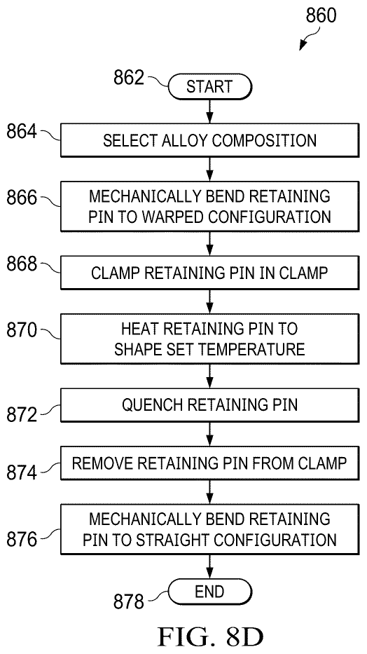

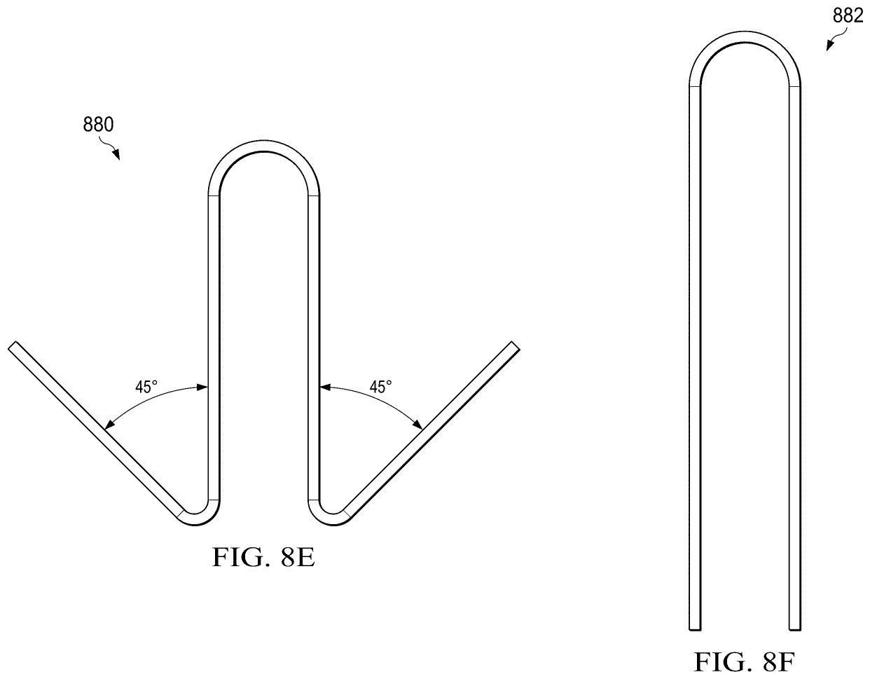

Referring then to FIGS. 8A and 8B, retaining block 402A will be further described.

Retaining block 402A is comprised of body 802. In a preferred embodiment, body 802 is generally cubical and is formed of suitable biocompatible plastic, such as previously described.

Body 802 generally has four equal planar square sides bounded by four equally spaced rounded vertical corners. Curvilinear slot 804 and curvilinear slot 806 are vertically disposed in body 802 and form an “x” pattern between the vertical corners. The curvilinear slots are generally planar and intersect perpendicularly at central contact gap 801. Each curvilinear slot proceeds downward in the body of the retaining block toward a semicircular floor. For example, curvilinear slot 806 terminates in semicircular floor 805B. Curvilinear slot 804 is similarly terminated in a similar semicircular floor, semicircular floor 805A. Retaining pin 108A resides within curvilinear slot 804. Likewise, retaining pin 108B resides in curvilinear slot 806. Each retaining pin generally forms an inverted “u” shape. Each retaining pin is secured within body 802 by epoxy 820. Contact gap 801 exists in epoxy 820 so as to allow electrical contact in the retaining pins, as will be further described.

Retaining pin 108A includes two straight sections 109A connected by a curved section 111A. Likewise, retaining pin 108B includes two straight sections 109B connected by a curved section 111B. The straight sections extend outward and downward from the base of body 802 between about 18 millimeters and about 22 millimeters. Retaining pin 108B contacts retaining pin 108A at apex fuse point 810. Retaining pin 108A is welded to retaining pin 108B at apex fuse point 810. The apex fuse point is used as an electrical contact between the retaining pins, as will be further described.

In one embodiment, the retaining pins are composed of about 20 to about 23-gauge nitinol alloy with a transition temperature of between about 30° C. and about 60° C. Nitinol, a nickel-titanium alloy, one of many shape-memory alloys, exhibits a shape memory that can “remember” its original shape and return to it after deformation, when exposed to certain stimulus, such as heat.

At lower temperatures, below the martensite finish temperature, “Mf”, nitinol exists in its martensite phase, which is soft and which can be deformed mechanically. Above the transition temperature, the so-called austenite finish temperature “A_{f}”, the nitinol reverts to its stronger austenite phase automatically recovering the programmed, warped shape. Transition between these phases enables the retaining pin to remember the warped shape when heated.

Referring then to FIG. 8D, preferred method 860 of preparing a retaining pin will be further described. In this example, the retaining pin is shape set to a transition temperature of about 37° C.

At step 862, the method begins.

At step 864, an alloy composition is selected. An untempered nitinol alloy composition of about 56% nickel and about 44% titanium, namely 55.8%, (atomic percent) nickel, and 44.2% titanium, is preferred for achieving a transformation temperature of approximately 37° C. Other alloys may be used in other embodiments. For example, cobalt based alloys, (Co—Cr—Ni), magnesium-based alloys (Mg—Sc), (Mg—Zn—Ca), platinum-based alloys (Pt—Ni) and gold-based alloys (Au—Cd) may all be employed. The nitinol alloy composition can also be changed to change the transition temperature. Increasing the nickel content will lower the transition temperature, while lowering the nickel content will raise the transition temperature. For example, decreasing the nickel content by 0.1% generally raises the transition temperature by 10° C. to 15° C. Hence, for a transition temperature of about 60° C., the alloy composition would be about 49.8% nickel and about 50.2% titanium.

Referring also to FIG. 8E, at step 866, at room temperature (e.g., about 22° C.), while the retaining pin is in its untempered state, it is mechanically bent to its “warped” or “high-temperature” configuration 880. In the warped configuration the straight sections of the retaining pin are bent to an approximate 45° angle upward. Other angles may also be usefully employed.

At step 868, the retaining pin, in its warped configuration, is fixed in a clamp to maintain its configuration during heat treatment.

At step 870, the retaining pin, is heat treated or “shape set” in its warped configuration. To shape set the retaining pin for a transition temperature of about 37° C., it is heated in a clean environment at a stress relief temperature of 500° C. Optionally, the heat treatment may be conducted in an argon environment to reduce oxidation. The stress relief temperature can range between about 400° C. and about 550° C. The heat treatment relaxes the internal stresses in the material caused from the mechanical deformation of bending the retaining pin into the warped state. For a 37° C. transition temperature, the preferred shape setting temperature is 500° C. as the internal stresses are received without causing over annealing. Using lower shape setting temperatures will raise the transition temperatures, while using higher shape setting temperatures will lower the transition temperature. For example, holding the shape setting temperature closer to 525° C. to 550° C. results in a transition temperature of about 60° C.

Preferably, the heat treat duration is about 10 minutes. Precise heating is preferred to avoid overheating or altering the material properties of the alloy. Increasing the time raises the transition temperature. Lowering the time lower the transition temperature. For example, for a 60° C. transition temperature, 15-30 minutes is preferred.

At step 872, retaining pin is quenched. Preferably, oil quenching is required for rapid coolant retaining the memory effect. The retaining pin is submerged in quenching oil for at least 10 minutes of about 30° C. After this step, the retaining pin in its tempered state having undergone the heat treatment process to receive internal stress and stabilize its properties.

At step 874, the retaining pin is removed from the clamp.

Referring also to FIG. 8F, at step 876, the retaining pin, while at room temperature, is mechanically bent into its “straight” or “low-temperature” configuration 882. The retaining pin will remain in its straight configuration at temperatures below the transition temperature. Hence, as long as the retaining pin is at room temperature, it will stay in the straight configuration.

At step 878, the method concludes.

To activate the shape memory of the alloy, the retaining pin is heated above the transition temperature. In this case, it can be provided by the internal body temperature of the patient, or an electric current, as will be further described. The retaining pin will retain its warped configuration so long as the temperature stays above the transition temperature of 37° C.

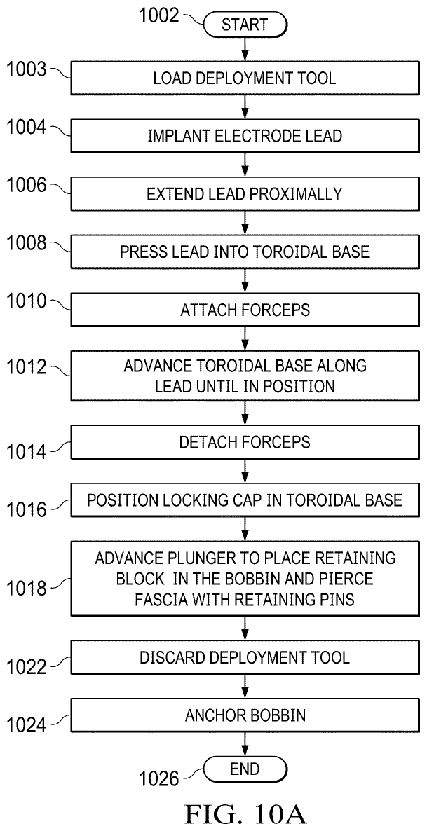

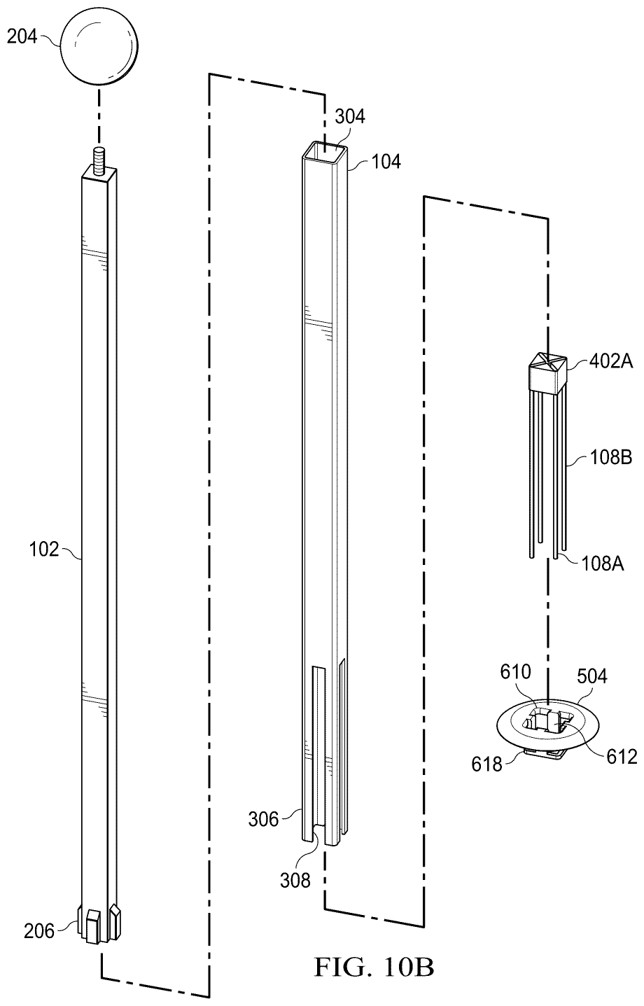

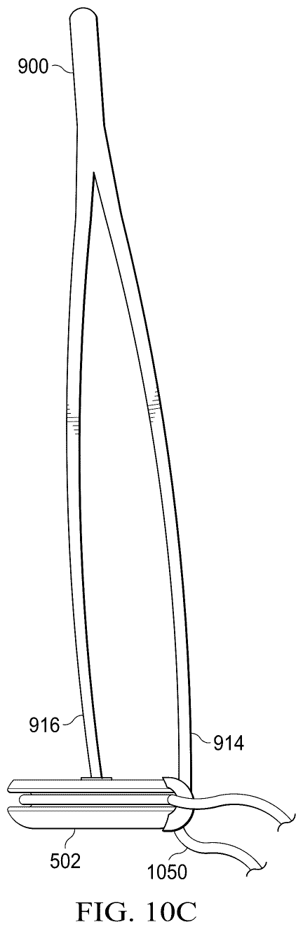

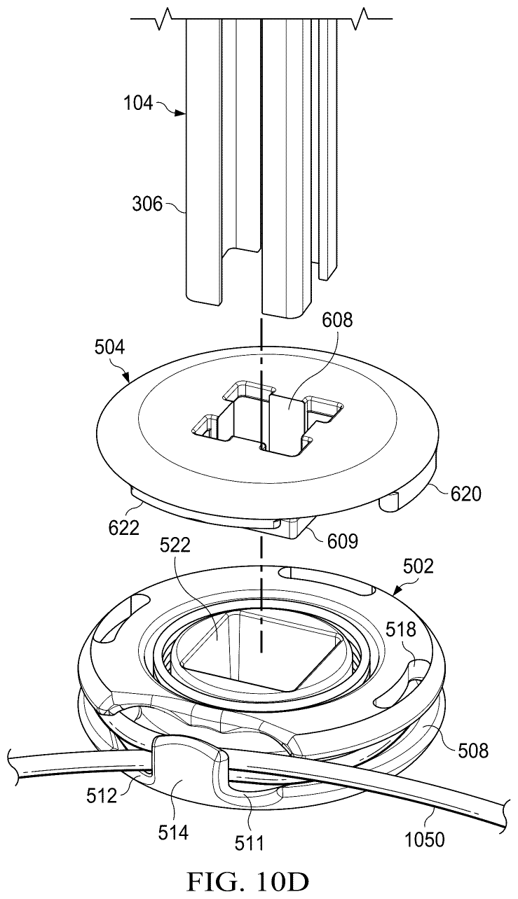

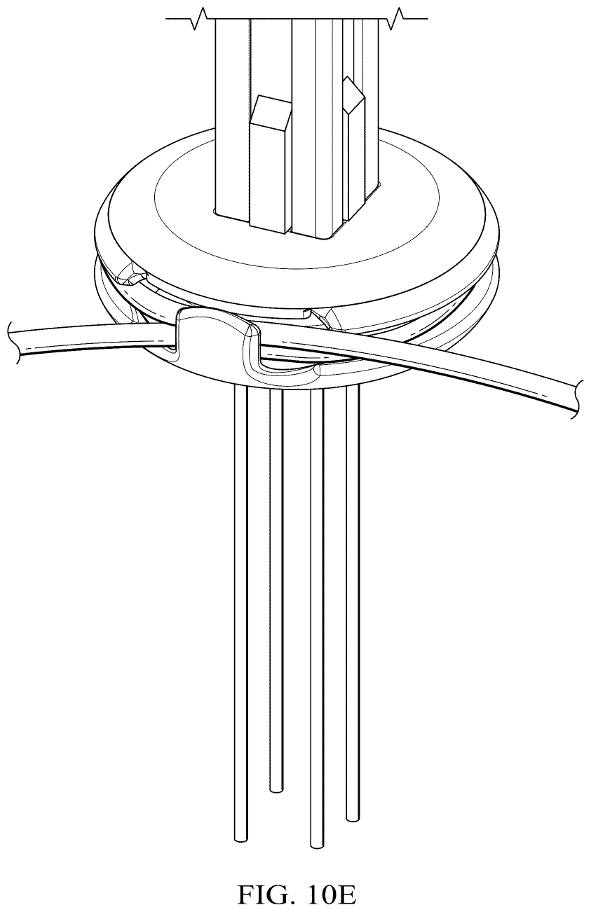

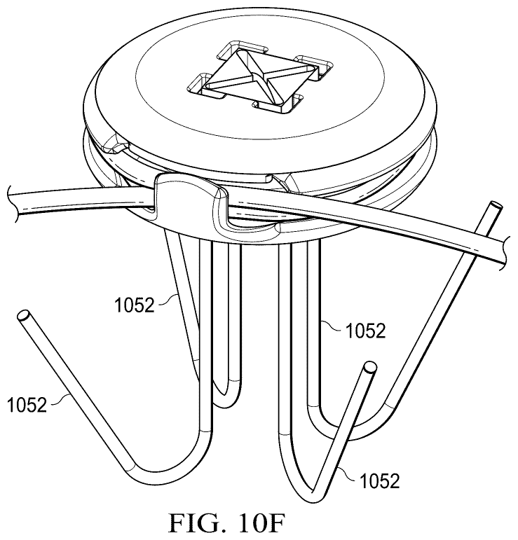

Referring then to FIGS. 8C and 10F, an alternate preferred embodiment of the retaining block and retaining pins will be further described.

Retaining block 402B includes retaining pins 108C and 108D. Each of the retaining pins is manufactured from medical grade stainless steel alloy wire such as type 316L or type 17-7PH of AWG24 gauge. Before deployment, each of the retaining pins is folded approximately midway in its length, at a bend such as bends 850. When deployed, the retaining pins automatically open, due to the elasticity of the stainless-steel material and automatically spring into the warped configuration. Other angles between about 20° and about 60° may be employed.

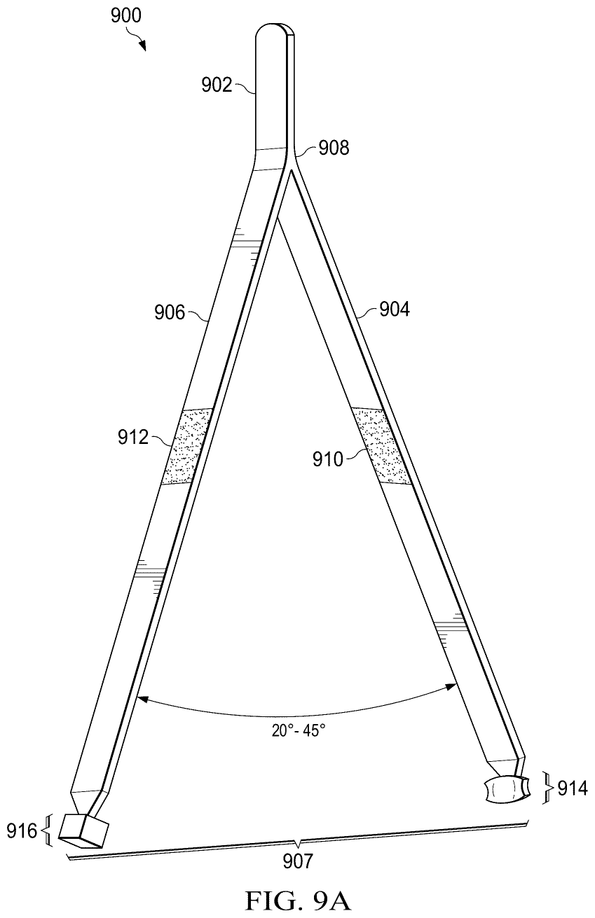

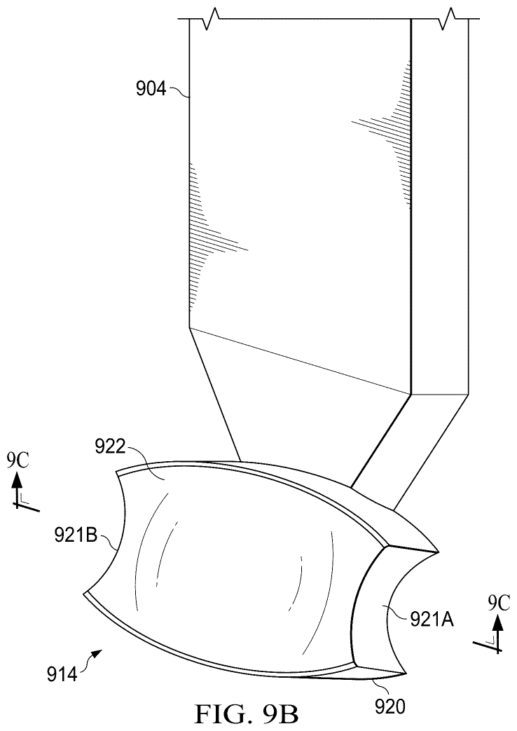

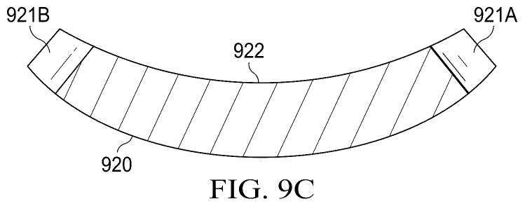

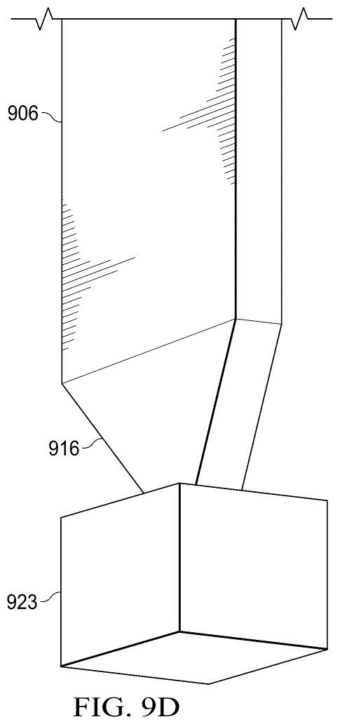

Referring then to FIG. 9A, forceps 900 will be further described. In general, forceps 900 are a medical grade pair of tweezers having specialized tips that are used to hold and position the toroidal base during the implantation procedure, as will be further described.

Forceps 900 include handle 902. Handle 902 further comprises arm 904 and arm 906, connected at spring joint 908. Spring joint 908 provides sufficient expansive force on each arm to maintain it at an approximate 20° to 45° extended angle, when the forceps is relaxed.

Arm 904 includes grip area 910. Arm 906 includes grip area 912. Both grip areas are centrally located on the exterior of the arm and can be rubberized or textured to improve comfort during use. Arm 904 includes tip 914. Arm 906 includes tip 916. Tip 914 and tip 916 form jaws 907 which are used in the procedure, as will be further described.

Referring to FIGS. 9B and 9C, tip 914 further includes crescent gripper 920. Crescent gripper 920 includes hemispherical surface 922 which is designed to generally match the oval circumference and dimensions of toroidal base 502. Hemispherical surface 922 includes opposing exit points 921A and 921B. The exit points each have a diameter slightly larger than the electrode lead so as to allow the lead to slide freely along the bobbin as the forceps are used, as will be further described.

Referring to FIG. 9D, tip 916 further comprises gripper cube 923. Gripper cube 923 is sized to fit within hole 522 of toroidal base 502, and in conjunction with crescent gripper 920, is used to hold toroidal base 502, and prevent it from turning during the implantation procedure, as will further described.

Referring to FIG. 10A, a preferred method of use will be further described.

At step 1002, the method begins.

At step 1003, the deployment tool is assembled and loaded. Referring also to FIG. 10B, plunger 102 is inserted into in center channel 304 of guide channel 104 from the bottom. Once the shaft is in place in the guide channel, button 204 is attached to the shaft. Locking tabs 206 are slidingly disposed in slots 308. A retaining block, such as retaining block 402A or 402B, is inserted into center channel 304, from the bottom upward, until it engages the base of plunger 102 adjacent locking tabs 206. In the case of retaining block 402A, the retaining pins are loaded in their tempered but straightened condition. In the case of retaining block 402B, the retaining pins are loaded in a retracted position such that they are spring loaded before deployment and automatically spring into shape upon deployment. Preferably, the retaining block is held in position within center channel 304 by a frictional interference between the retaining pins and the inside corners of connector posts 306. Once in position, the retaining pins are positioned adjacent the base of guide channel 104 and directly above the triangular exit holes. The connector posts are placed in the positioning indentions of locking cap 504 adjacent positioning posts 612. The connector posts are held in the positioning indentions by a friction fit between the connector posts and positioning indentions. Connector posts 306 extend downward adjacent to and in contact with perimeter wall 614 of locking cap 504.

At step 1004, the electrode lead is implanted in the patient, as is known in the art. In one embodiment, the electrode lead is implanted in the spine, as previously described.

At step 1006, the implanted lead is extended proximally away from the patient.

At step 1008, the implanted lead, such as example lead 1050, is impressed into annular slot 508 of toroidal base 502. As can be seen most clearly in FIG. 10D, the lead is wrapped at least once around the bobbin in the annular slot, entering at lead opening 512 and exiting at lead opening 511, adjacent lead guide post 514. In this example, only one turn of the lead is impressed in the annular slot. However, in other embodiments, the annular slot may be sized to accommodate multiple turns of the lead. In each case, lead guide post 514 serves to hold the lead in the annular slot by virtue of being pressed past the annular gap formed by retaining lips into the larger diameter annular slot. Importantly, wrapping the lead around the bobbin positions at least one turn of the lead in longitudinal central axis 503 of the bobbin, as can be seen in FIG. 5C, and so form the primary coil of the air coil transformer.

Referring also to FIG. 10C, at step 1010, forceps 900 are attached to the toroidal base by impressing tip 916 into hole 522. The arms of the forceps are compressed, positioning tip 914 adjacent lead guide post 514, thereby pressing hemispherical surface 922 of crescent gripper 920 against lead guide post 514. In this way, the crescent gripper holds the lead within lead opening 511 and lead opening 512.

At step 1012, the toroidal base is moved along the lead. In practice, lead 1050 slides or “rotates” annularly along lead 1050, within the annular slot, so that toroidal base 502 may be moved linearly along the length of the lead without disturbing implanted electrode.

At step 1014, the forceps are detached from the toroidal base, by releasing pressure on the arms and removing tip 916 from the toroidal base.

Referring also to FIGS. 10D and 10E, at step 1016, the loaded deployment tool is used to position the locking cap in the toroidal base. Cuboidal feature 609 of locking cap 504 is positioned downward into hole 522 of toroidal base 502. Arcuate locking pins 620 are positioned in locking slots 518, and arcuate retainer tab 622 is positioned in arcuate indention 507, downward against lead guide post 514, thereby holding lead 1050 in lead opening 511 and lead opening 512. At the same time, lower surface 606 is securely and flatly positioned against top surface 516. The accurate locking pins extend vertically downward into annular slot 508 and press against exterior surface of lead 1050 thereby compressing it against the annular slot and preventing it from slipping in the annular slot.

At step 1018, button 204 is depressed, thereby advancing the plunger such that the retaining block is forced downward in center channel 304 of guide channel 104, guided by locking tabs 206 sliding in guide slots 308, until the retaining pins protrude through triangular exit holes 618 and enter the fascia. Retaining block 402 is advanced until it is securely seated against base 616 in the cuboidal feature. At the same time, arcuate locking pins 620 are locked in position in locking slots 518. In one embodiment, the positioning posts of the cuboidal feature are sized so that an interference fit exists between the inside surfaces of the positioning posts and the exterior of body 802 of retaining block 402, thereby preventing the retaining block from exiting hole 608 while the retaining pins are deployed. In another embodiment, the retaining block is held in the cuboidal feature and the locking cap is held in the toroidal base by the force exerted by the retaining pins when they are deployed in the fascia, as will be further described.

As step 1022, the exhausted deployment tool, automatically detached from the assembled bobbin, is discarded.

Referring to FIG. 10F, at step 1024, the bobbin is anchored in the fascia by warping retaining pins 1052. In other embodiments, the bobbin can be anchored to other anatomical structures that offer sufficient mechanical support, depending on the specific implantation site and therapeutic needs. These structures may include the periosteum of bones such as the vertebrae or ribs, tendinous attachments near the spine like the supraspinous or interspinous ligaments, and the robust thoracolumbar fascia. Additionally, muscle sheaths such as the epimysium of paraspinal muscles, ligaments including the ligamentum flavum or sacrospinous ligament, and even subcutaneous tissues with firm underlying attachments may serve as anchoring sites. For interventions targeting upper thoracic or cervical regions, scapular attachments could also be utilized. Each anchoring site is selected to ensure positional stability of the bobbin while minimizing any potential interference with surrounding tissues or functionality of the implanted lead system.

In one embodiment, the retaining pins are shape set with a transition temperature at or above 37° C. In order to transition the retaining pins into their warped configuration, the apex of retaining pin 108A is briefly brought in contact with a monopolar cautery (not shown), also known as a monopolar electrosurgical instrument. The instrument produces a high-frequency electrical current which typically allows for surgical cutting or coagulation. However, in this case, the current is used to raise the retaining pins to their transition temperature. In practice, retaining pin 108A is briefly contacted by the instrument, through contact gap 801, producing ohmic heating between the straight sections and the adjacent coagulating tissue. The ohmic heating exceeds the transition temperature sufficient to return the retaining pins to their warped configuration.

In another embodiment, the retaining pins are shape set with a transition temperature of between about 35° C. and about 37° C. When exposed to body temperature, which meets or exceeds the transition temperature, the pins transition from their straight configuration to their warped configuration within the fascia.

In yet another embodiment, the retaining pins are warped before insertion into the deployment tube and “spring” into position mechanically (i.e., without a material phase change) upon deployment when the fascia is pierced.

In all cases, a spring force against the body tissue is created by the warped shape of the retaining pins, which has the desired effect of anchoring the bobbin in place. Once the bobbin is anchored, retaining block is held securely in hole 608, and the device is permanently anchored to the fascia or other body structure.

At step 1026, the method ends.

In yet other embodiments, positional anchors, such as those described in U.S. application Ser. No. 17/443,174 to Wolf II, may be employed in place of the retaining pins. Likewise, the retaining pins as described can be employed with the anchors shown and described in Wolf II.

1. An anchor device configured to anchor an electrode lead to a bodily structure, the electrode lead configured to deliver electrical stimulation to a patient, the anchor device comprising:
a toroidal base;
an annular slot, formed in a circumference of the toroidal base;
the electrode lead removably positioned in the annular slot, forming a primary coil of an air-core transformer;
a cylindrical cavity axially formed in the toroidal base;
a locking cap receiver, axially formed in the toroidal base;
an electrical coil, operably fixed in the cylindrical cavity, forming a secondary coil of the air-core transformer;
the air-core transformer creating a mutual inductance between the primary coil and the secondary coil;
a locking cap, removably fixed in the locking cap receiver; and
a retaining means, fixed to the anchor device, for securing the anchor device to the bodily structure.

2. The anchor device of claim 1, wherein the air-core transformer raises an inductance value of the electrode lead.

3. The anchor device of claim 1, wherein the retaining means further comprises:
a retaining block receiver, axially formed in the locking cap;
a retaining block, lodged in the retaining block receiver;
a set of retaining pins, projecting from the retaining block; and
whereby the anchor device is secured to the bodily structure by the set of retaining pins.

4. The anchor device of claim 3:
wherein the set of retaining pins is comprised of a shape memory alloy; and
wherein the shape memory alloy transitions from a low-temperature shape to a high-temperature shape at a transition temperature;
wherein the transition temperature is between about 35° C. and about 60° C.

5. The anchor device of claim 4, wherein the low-temperature shape is approximately straight and the high-temperature shape is warped.

6. The anchor device of claim 4, wherein the transition temperature is about 37° C. and is provided by the bodily structure.

7. The anchor device of claim 4, further comprising a monopolar cautery instrument, in electrical contact with the set of retaining pins, whereby the transition temperature is reached.

8. The anchor device of claim 3, wherein the electrical coil further comprises a shortened coil having a plurality of windings.

9. The anchor device of claim 8, wherein:
the primary coil has a first transverse central plane;
the secondary coil has a second transverse central plane; and
the first transverse central plane is coplanar with the second transverse central plane.

10. The anchor device of claim 3, wherein the toroidal base further comprises:
a set of longitudinal locking slots impinging on the annular slot;
the locking cap further comprises a set of longitudinal locking pins, positioned in the set of longitudinal locking slots, so as to contact the electrode lead in the annular slot; and
whereby the electrode lead is fixed in the annular slot relative to the toroidal base.

11. The anchor device of claim 3, wherein the annular slot further comprises:
a set of lead openings, separated by a lead guide post;
the locking cap further comprises a retainer tab, positioned in the set of lead openings; and
whereby the electrode lead is prevented from escaping the annular slot.

12. The anchor device of claim 3, wherein:
the locking cap receiver is a rectangular hole; and
the locking cap further comprises a cuboidal feature positioned in the rectangular hole.

13. The anchor device of claim 12, wherein:
the cuboidal feature further comprises a set of positioning indentions separated by a set of longitudinal positioning posts integrally formed with a perimeter wall; and
whereby a deployment tool may be coupled to the cuboidal feature.

14. The anchor device of claim 13, wherein:
the retaining block is lodged in the cuboidal feature adjacent the set of longitudinal positioning posts; and
the set of retaining pins protrude outside the toroidal base.

15. The anchor device of claim 1, wherein the locking cap further comprises:
a retaining block receiver;
the retaining block receiver removably fixed to a set of forceps;
the set of forceps further comprising a rectangular tip positioned in the retaining block receiver and a crescent tip impressed against an external surface of the toroidal base and at least partially covering the annular slot; and
whereby the electrode lead is retained in the annular slot.

16. The anchor device of claim 1, wherein the locking cap further comprises:
a retaining block receiver;
the retaining block receiver removably fixed to a deployment tool; and
the deployment tool further comprising:
a guide channel having a center channel, removably fixed to the retaining block receiver;
a plunger, slidingly disposed in the guide channel, having a loaded configuration and a deployed configuration;
the loaded configuration further comprising of retaining block, having a set of retaining pins, slidingly disposed in the guide channel, adjacent to the plunger; and
the deployed configuration further comprising the retaining block positioned in the retaining block receiver, adjacent to the plunger, and the set of retaining pins protruding from the toroidal base.

17. The anchor device of claim 16, wherein the guide channel has a first square cross-section and the plunger has a second square cross-section.

18. A method of implanting an anchor device, for an electrode lead, to a bodily structure, the electrode lead configured to deliver electrical stimulation to a patient, comprising:
providing the anchor device with a toroidal base, adapted to receive a locking cap;
pressing the electrode lead into a circumferential slot of the toroidal base, such that the electrode lead forms a primary coil of an air-core transformer;
providing the toroidal base with an axially located electrical coil, interior to the circumferential slot, such that the axially located electrical coil forms a secondary coil of the air-core transformer;
fixing the locking cap to the toroidal base, such that the locking cap prevents escape of the electrode lead from the toroidal base; and
anchoring the toroidal base and the locking cap to the bodily structure.

19. The method of claim 18, wherein the step of anchoring further comprises:
providing a retaining block, having a set of extended retaining pins;
positioning the retaining block in the locking cap, such that the set of extended retaining pins extend from the toroidal base and into the bodily structure; and
warping the set of extended retaining pins.

20. The method of claim 19, wherein the step of positioning further comprises:
providing a deployment tool, having a loaded configuration and a deployed configuration;
attaching the deployment tool to the locking cap; and
moving the deployment tool from the loaded configuration to the deployed configuration, thereby moving the retaining block from the deployment tool to the locking cap.

21. The method of claim 19, wherein the step of warping further comprises:
raising a temperature of the set of extended retaining pins past a transition point.

22. The method of claim 21, wherein the step of raising further comprises:
applying a monopolar cautery instrument to the set of extended retaining pins.

23. The method of claim 18, further comprising the step of:
sliding the toroidal base along the electrode lead without removing the electrode lead from the toroidal base.

24. The method of claim 20, further comprising the step of:
detaching the deployment tool from the locking cap.

25. An anchor device configured to anchor an electrode lead to a bodily structure, the electrode lead configured to deliver electrical stimulation to a patient, the anchor device comprising:
a rounded anchor body;
a clamping means for securing the electrode lead in the rounded anchor body;
a set of retaining pins projecting from the rounded anchor body; and
whereby the set of retaining pins is comprised of a shape memory alloy having a transition temperature between about 35° C. and about 60° C.

26. The anchor device of claim 25, wherein the transition temperature is about 37° C.

27. The anchor device of claim 25:
wherein the set of retaining pins have a set of anchor sections for contacting the bodily structure; and
wherein the set of anchor sections has a low-temperature shape and a high-temperature shape;
wherein the set of anchor sections changes from the low-temperature shape to the high-temperature shape at about the transition temperature.

28. The anchor device of claim 27:
wherein the low-temperature shape is straight;
wherein the high-temperature shape is warped so as to form an anchor within the bodily structure.

29. A method of preparing a retaining pin for an anchor device, the anchor device for use in securing an electrode lead to a bodily structure of a patient, the method comprising:
forming the retaining pin from a shape memory alloy;
mechanically bending the retaining pin into an anchor shape while the shape memory alloy is in a martensite phase;
fixing the retaining pin in the anchor shape using a mechanical clamp;
heating the retaining pin above a stress relief temperature for a finite time;
quenching the retaining pin thereby fixing it in an austenite phase;
removing the retaining pin from the mechanical clamp; and
mechanically bending the retaining pin into a deployment shape for piercing the bodily structure while the shape memory alloy is in the austenite phase.

30. The method of claim 29, wherein the stress relief temperature is about 500° C.

31. The method of claim 29, wherein the finite time is about 10 minutes.

32. The method of claim 29, wherein the shape memory alloy is nitinol having a composition of about 56% nickel and about 44% titanium.
